(19) United States
(12) Patent Application Publication
(10) Pub. No.: US 2004/0047872 A1
(43) Pub. Date: Mar. 11, 2004

(54) Indicators for monitoring the technique of transcutaneous immunization

(75) Inventors: Gregory M. Glenn, Cabin John, MD (US); Carl R. Alving, Bethesda, MD (US)

Correspondence Address: MORGAN, LEWIS & BOCKIUS L.L.P., 1111 Pennsylvania Avenue, N.W., Washington, DC, 20004, US

(73) Assignee: The Government of the United States, as represented by the Secretary of the Army

(21) Appl. No.: 10/658,418

(22) Filed: Sep. 10, 2003

Related U.S. Application Data
(63) Continuation of application No. 09/585,559, filed on Jun. 2, 2000, now abandoned.
Application No. 09/585,559 is a continuation-in-part of application No. 09/257,188, filed on Feb. 25, 1999.
Application No. 09/257,188 is a continuation-in-part of application No. PCT/US97/21324, filed on Nov. 14, 1997.
Application No. PCT/US97/21324 is a continuation-in-part of application No. 08/896,085, filed on Jul. 17, 1997, now Pat. No. 5,980,898.
Application No. 08/896,085 is a continuation-in-part of application No. 08/749,164, filed on Nov. 14, 1996, now Pat. No. 5,910,306.
(60) Provisional application No. 60/137,790, filed on Jun. 3, 1999.

(51) Int. Cl. A61K 39/00

(52) U.S. Cl. 424/184.1

(57) ABSTRACT

Transcutaneous immunization system delivers antigen to immune cells through topical application, and induces an antigen-specific immune response in an animal or human. The improvement involve a system for marking the successful delivery of the immunization using indicators or using marking systems to allow better application of the patch to a prepared skin surface.

### BACKGROUND OF THE INVENTION

1. Field of the Invention

The invention relates to transcutaneous immunization and the use of markers to identify animals and humans that have been vaccinated or to assist in the process of immunization.

2. Description of the Related Art

Immunization is one of the most effective medical interventions and has changed the face of both human and animal health. Compliance with vaccines can be an important problem and successful vaccination requires adherence to proper technique. Extensive records are kept for patients ensuring proper compliance with vaccination schedules. Transcutaneous immunization using the skin may be no different from other methods of vaccination in that the successful immunization will require proper technique for delivery of the vaccine antigen.

Topical application of a vaccine antigen may present particular problems unique to the TCI method as, unlike intramuscular immunization where the complete vaccinating solution is injected and enclosed within the tissue and will not be brushed away, it is conceivable that a topical application could be brushed off or otherwise lost. An indicator associated with or within the immunizing solution would allow the delivery to be monitored for proper technique.

Skin, the largest organ of the human body, is an important part of the body's defense against invasion by infectious agents through its well described barrier function (see Bos, 1997, Fisher's “Contact Dermatitis”). Vibrio cholera and cholera toxin (CT) are examples of infectious agents and their products, respectively, which one would have expected the skin to protect against. In fact cholera toxin, once through the skin, is well known to be noxious. Craig (1965) reported that stool filtrates of cholera patients injected intracutaneously into rabbits or guinea pigs produced a characteristic delayed, sustained edematous induration (swelling), which was induced by the presence of toxin in the skin. The swelling and vascular leakage was so dramatic that it was ascribed to an unknown permeability factor which was later shown to be CT itself. Similarly, as little as 5 ng of CT injected into the skin can cause local redness and swelling. In our laboratories, we have found that injection of CT into the muscle bed in even small amounts causes severe swelling and even death in immunized animals. Thus, one could have reasonably expected that CT would be extremely reactogenic when placed on the skin, and cause similar redness induration, tenderness and swelling. The reactogenicity produced by CT injected through the skin, the “Craig test”, became a standard measurement for the presence and amount of CT in stool filtrates or culture media. Data confirmed that this skin reactivity was due to cholera toxin (see Finkelstein and LoSpallutto, 1969). As a result, Craig (1965) cautioned, “The absence of skin lesions in clinical cholera certainly does not preclude the possibility that the noxa responsible for gut damage could also have a deleterious effect upon the skin provided it is applied to skin in sufficient concentration”. The extreme reactogenicity of cholera toxin in the skin was used as a test for its toxicity and such prior art evidenced an expectation that cholera toxin would be highly reactogenic if applied to the skin, producing similar swelling and redness if it were to penetrate the skin.

In contrast, we have shown cholera toxin to be immunogenic, acting as both antigen and adjuvant, when placed on the skin but without systemic side effects (U.S. application. Ser. No. 08/749,164 (filed Nov. 14, 1996); U.S. application Ser. No. 08/896,085 (filed Jul. 17, 1997); and international application PCT/US97/21324 (filed Nov. 14, 1997). This lack of systemic reactogenicity when cholera toxin was placed on the skin for transcutaneous immunization was surprising and contradicted conclusions one would have drawn from the prior art. Specifically, CT placed on the skin according to our invention acts as a non-toxic, non-reactogenic adjuvant, in contrast to the expectations of Craig, while injection of CT into the skin results in severe swelling and redness and use of CT by the oral and nasal routes induce systemic side effects. Thus, it was not obvious prior to our invention that cholera toxin or other ADP-ribosylating exotoxins or other adjuvants would be useful for transcutaneous immunization. Further, it is not obvious that such transcutaneous immunizations could be placed on the skin surface and provided with an indicator which would permit monitoring of the proper use of this novel immunization technique.

In many cases, effective immunization that leads to protection and requires help in the form of adjuvants and therefore useful immune responses requires the use of an adjuvant to enhance the immune response. (N.E.J.M. 1997, Vol. 336; p. 86-91) Generally, vaccine antigens are mixed or complexed by adjuvants to enhance the induction of an immune response and, in the absence of adjuvants, the immune response is generally inadequate to sufficiently stimulate an immune response. In international application PCT 0597/21324 we show the principal that a skin adjuvant can induce high levels of systemic and mucosal antibodies to coadministered antigens. For example, mice immunized with CT+DT induced high levels of systemic and mucosal anti-DT antibodies. Antibodies are known to be the immune correlate for protection against diphtheria. Thus adjuvants for transcutaneous immunization can be expected to provide ‘help’ in the immune responses to coadministered antigens and play a critical role in a useful immune response. As described below, many compounds and biological products may act as adjuvants on the skin, possibly targeting the associated APCs such as Langerhans cells or dermal dendritic cells or draining lymph node cells to induce an immune response.

However, the previous references with respect to penetrability and size explain why our successful use of a molecule like cholera toxin (which is 86,000 daltons) as an antigen adjuvant in immunization was greeted with surprise by the art because such large molecules were not expected to pass through the skin and, therefore, would not have been expected to induce a specific immune response. However, we have shown in U.S. application Ser. No. 08/749,164 (filed Nov. 14, 1996); U.S. application Ser. No. 08/896,085 (filed Jul. 17, 1997); and international application PCT/US97/21324 (filed No. 14, 1997) that using an ADP-ribosylating exotoxin, such as cholera toxin, as an antigen could elicit a vigorous immune response which was highly reproducible. When an ADP-ribosylating exotoxin, such as cholera toxin, was used as an immunoadjuvant and applied to the skin in a saline solution with a separate antigen (e.g., bovine serum albumin, diphtheria toxoid), a systemic and mucosal antigen-specific immune response could be elicited. We have shown that like cholera toxin, heat-labile enterotoxin from E. coli (LT), Pseudomonas exotoxin A (ETA), and pertussis toxin (PT) are able to pass through the skin and induce an immune response when present in a transcutaneously applied formulation. Additionally CT, LT, ETA and PT can act as adjuvants to induce an immune response to antigens coadministered on the skin. Thus most antigens, not highly immunogenic by themselves when applied transcutaneously to the skin, can induce a strong immune response when placed on the skin with CT or other adjuvants. It has been shown in our lab that other adjuvants such as LPS, lipid A, TNFa, GMCSF, could similarly be expected to pass through the skin if the skin is adequately hydrated. (Kersten et al.)

The Langerhans cell population underlying the site of application are a preferred antigen presenting cell for delivering antigen to the immune system, although other dendritic cells, macrophages, Kupffer cells or B-cells may be targeted as well. Adjuvant may act on the antigen presenting cell directly, or indirectly through bystander effects, or through cognate lymphocytes specifically recognizing antigen.

### SUMMARY

This present invention is applicable to the immunization of both humans and lower animals including mammals and birds. A particular problem that may arise from the transcutaneous immunization of human and animal subjects is the identification of which subjects have been immunized. As the topical immunization may not raise any local swelling or local induration, there may be no external marker for confirming that the immunization has taken place. Immunizations of lower animals requires the use of some form of identifier due to the lack of ability for the animal to communicate. Immunization of humans can also present significant identification problems in situations where the immunized human subject is unable to communicate important information relative to the immunization, for example: when was the immunization done, with what formulation, for what purpose, etc. Such situations obviously would arise when transcutaneously immunizing children, adults of limited communication ability due to foreign language, or age, or level of consciousness due to illness, and the like.

The present invention discloses compositions, articles combined with such compositions, and methods of use of such compositions and articles which give observable indications related to the specific immunization, time span of the immunization application, effectiveness of the immunization, and other parameters related to the practice of transcutaneous immunization according to the present invention.

### DETAILED DESCRIPTION OF THE PRESENT INVENTION

The following description of preferred and alternative embodiments of the present invention is provided as a non-limiting disclosure of the broad concept of the present invention of providing effective transcutaneous immunization to humans and animals such that at least one indicator is provided therewith; the indicator/s providing a wide variety of information relating to the characteristics and effectiveness of the administered immunization.

Immunizations of lower animals obviously require the use of some form of identifier due to the lack of communication. For example, if production animals such as cows, pigs and sheep may be mass immunized by passing through a gate. It would be useful to mark the animals with a dye or coloring that indicates that the animals have been immunized. Similarly, it may be useful to mark the immunization site before application. This is especially true if the site is to be cleaned or treated in nay way before immunization. For standard vaccination by the intramuscular route, this cannot usually be done as injection of a dye might spoil the carcass. As the skin is usually discarded, marking, tattoos, branding etc. that is limited to the hide would be of no commercial detriment. Marking the immunized animals collected in a holding pen would both ensure that all animals are immunized and ensure that no animals are immunized more than once.

In human applications, it is conceivable that a patch or gel or cream or other suitable vehicle will be applied to the vaccines. If the vehicle contains an indicator such as a dye, then the successful application of the immunizing solution, patch, gel, emulsion or other delivery vehicle can be monitored. For example, if a child were given a patch which after proper application left an indicator such as a dye or temporary tattoo (cartoon figure for example), then the proper application could be better ensured. It may also arise that a timed release of indicator could ensure that the immunizing solution, patch, gel emulsion or other delivery vehicle is applied for adequate period of time to ensure proper immunization. It is conceivable that the immunization requires a minimum of time of application and that the indicator or dye could be triggered to be released after a certain time of application. Alternatively, other triggers such as adequate hydration may induce the presence of an indicator onto the skin to indicate that the conditions for immunization have been properly met. Thus, when the immunization is considered complete, the immunizing solution, patch, gel emulsion or other delivery vehicle may turn color and be discarded.

There may be a need to cause destruction of the adjuvant or antigen after the minimum time of immunization is completed. For example, the use of a CT holotoxin may not be aceptable in cases where the immunizing solution is never washed off. If the immunization solution contained a timed release microcapsule or other vehicle that released enzymes, acids, or bases that deactivates the adjuvant or antigen, then the environmental hazard can be eliminated and its elimination confirmed with a concurrent color change. Thus, when the patch or skin turns color, the patch may be safely discarded.

The antigen or adjuvant or skin activator or indicator may assist in the passage through the stratum corneum and promote contact with immune cells. For example, the indicator as described herein may promote contact between an antigen-adjuvant and an antigen presenting cell of the immune system (e.g., Langerhans cells in the epidermis, dermal dendritic cells, follicular dendritic cells, macrophages, B cells) and/or induce the antigen presenting cell to take up the antigen-adjuvant; the antigen presenting cell would then present the antigen to a lymphocyte. In particular, the antigen presenting cell may migrate from the skin to the lymph nodes, and then present antigen to a lymphocyte, thereby inducing an antigen-specific immune response. Moreover, the formulation may directly contact a lymphocyte which recognizes antigen, thereby inducing an antigen specific immune response. In addition to eliciting immune reactions leading to activation and/or expansion of an antigen-specific B and/or T cell population, including a cytotoxic T lymphocyte (CTL), another object of the invention is to positively and/or negatively regulate components of the immune system by using the transcutaneous immunization system to affect antigen-specific helper (Th1 and/or Th2) or delayed-type hypersensitivity (DTH) T-cell subsets. This can be exemplified by the differential behavior of CT and LT which can result in different T-helper responses. It would be expected that these immune responses could lead to protective immune responses such as anti-tetanus toxoid antibodies for tetanus or anti-diphtheria antibodies for diphtheria.

In one embodiment of the invention, a formulation containing antigen and adjuvant is applied to intact skin of an organism, the antigen is presented to immune cells, and an antigen-specific immune response is induced by applying the immunizing solution, patch, gel emulsion or other delivery vehicle to the skin with an indicator. The formulation may include additional antigens such that transcutaneous application of the formulation induces an immune response to multiple antigens. In such a case, the antigens may or may not be derived from the same source, but the antigens will have different chemical structures so as to induce immune responses specific for the different antigens. Antigen-specific lymphocytes may participate in the immune response and, in the case of participation by B lymphocytes, antigen-specific antibodies may be part of the immune response; alternatively cytotoxic T-cells specific for the antigen may be induced.

In another embodiment of the invention, the invention is used to treat an organism. If the antigen is derived from a pathogen, the treatment vaccinates the organism against infection by the pathogen or against its pathogenic effects such as those caused by toxin secretion. A formulation that includes a tumor antigen may provide a cancer treatment; a formulation that includes an autoantigen may provide a treatment for a disease caused by the organism's own immune system (e.g., autoimmune disease). A formulation that contains an allergen may be used for immunotherapy. The invention may be used therapeutically to treat existing disease, protectively to prevent disease, or to reduce the severity and/or duration of disease. In a further embodiment of the invention,a patch for use in the above methods is provided. The patch may comprise a dressing, and effective amounts of antigen and adjuvant.

The dressing may be occlusive or non-occlusive. The patch may include additional antigens such that application of the patch induces an immune response to multiple antigens. In such a case, the antigens may or may not be derived from the same source, but the antigens will have different chemical structures so as to induce an immune response specific for the different antigens. Multiple patches may be applied simultaneously; a single patch may contain multiple reservoirs. Each reservoir may contain its own indicator as application conditions and successful immunization conditions may be individual. For effective treatment, multiple patches may be applied at frequent intervals or constantly over a period of time, (See U.S. Pat. No. 5,049,387 for a detailed description of a patch) or may be applied simultaneously. Creams, ointments, gels and other vehicles may be applied in a similar fashion using multiple antigens and adjuvants both at the same or separate sites or simultaneously or in frequent, repeated applications, each with its own unique indicator or the same indicator. The patch may include a controlled release reservoir or a matrix or rate controlling membrane that may be used which allows stepped release of antigen, adjuvant or indicator. The patch may contain a single reservoir with antigen and adjuvant or multiple reservoirs with individual antigens and adjuvants and indicators. The immunization may be conducted by first placing the antigen at the site and, at some other time or some other site, adding the antigen. The order of application may be reversed.

The site may be protected with anti-inflammatory corticosteroids such as hydrocortisone, triamcinolone and mometazone to reduce possible local skin reaction. Similarly anti-inflammatory steroids and compounds may be included in the patch material, in creams, ointments, etc. or such compounds may be applied after immunization. Although anti-inflammatory steroids are generally used to deplete Langerhans cells, we have found that immunization using the skin could be conducted after the majority of LCs were depleted by the application of topical anti-inflammatory steroid. The antiflammatory cream may be mixed with an indicator to demonstrate its presence if some immunizing preparations require hydrocortisone or other additives to indicate its presence. The site may be pretreated with a depilatory such as calcium hydroxide. The skin may be swabbed with alcohol as is standard for injectable vaccination. The skin may be swabbed before immunization with an indicator which will then be absorbed, or washed away into the immunizing solution, patch, gel emulsion or other delivery vehicle at the completion of immunization. The skin may be pretreated with alcohol or acetone and indicator for a time period prior to immunization known to increase the number of Langerhans cells in the skin to enhance the immunization.

Moreover, in yet another embodiment of the invention, the formulation is applied to intact skin overlying more than one draining lymph node field using either single or multiple applications. The formulation may include additional antigens such that application to intact skin induces an immune response to multiple antigens. In such a case, the antigens may or may not be derived from the same source, but the antigens will have different chemical structures so as to induce an immune response specific for the different antigens. Different indicators may be associated with each particular antigen or adjuvant to indicate its presence or demonstrate proper application technique. The formulation may be applied to intact skin to boost or prime the immune response and different indicators may be used for the prime and boost. The indicator may be activated by penetration through the stratum corneum, contact with living keratinocytes or immune cells such as Langerhans cells. The indicator may be combined with the adjuvant or antigen or vehicle.

The indicator could be the color of the patch, a dye such as employed in food color, ink, vital stain, Evans blue, paint, natural colorings,. oxides, chlorophyll, charcoals, chalks, powders, pH indicators, peroxidase triggered or enzyme triggered, florescent or other agents visible using UV light, radioactively tagged, attached to beads, gold particles, or could be indicated by induction of an odor (smell). The indicator may be in the form of a reporter gene such as green florescent protein or luciferase, incorporated into a plasmid.used for immunization.

When an immunization is to be performed, there may need to be a method for marking the site. A convenient marking system and effective penetration enhancing step may be combined. For example, tape stripping, the application of a commonly purchased adhesive tape such as (e.g. Scotches tape) can be applied to the skin and removed. When the tape is removed a layer of stratum comeum is also removed. This step may be repeated many times, even to the point of removing Langerhans cells. It is envisioned that a piece of adhesive tape with marking ink or other suitable substance can be applied in such a manner as to both mark the site to be immunized and tape strip the skin to enhance penetration and therefore enhance immunization. The tape and its marking can be manufactured in such a way as to delineate the exact area to be immunized. This will allow the nurse or other person administering the immunization to apply the immunizing patch or vehicle to the site that has been prepared. Preparation of the site might also involve tape stripping, marking followed by alcohol swabbing or the use of other agents to remove lipids and dead cells such as acetone, depiliators, detergents or even, water.

The tape strip/marking can be performed in a wide variety of ways, including several sites with different color coded markings for specific vaccines; aligned by using a letter in the ink printed on the subject that matches a letter on the patch so that the adhesive may be specifically adjusted for the particular vaccine; at multiple sites; for designer patches with specific vaccines or allergens; for single or multiple use; in a table top, hand held or attachable or pocket dispenser.

In another embodiment of the invention, an air powered gun such as a paint gun, may be employed to deliver the vaccine to the surface of the skin. For example range cattle or wild animals that cannot be easily captured may be shot with a plastic bag containing the antigen, adjuvant, penetration enhancer, adhesives and indicator or any combination of the above. Thus, the immunized animals so targeted can be marked as immunized. There are a variety of propulsion mechanisms that can be employed including for example, CO2 or helium powered guns, or pump air-guns. The velocity of the projectile may be tailored for the animal species to be targeted so as to enhance the penetration; some animals may merely require surface application of the immunizing solution. Others might be better immunized if a superficial lesion was generated at the site of immunization by the projectile.

It is possible that the application of the immunizing solution is preferably licked off or ingested or inhaled by an animal. This may either enhance the transcutaneous immunization by providing an additional route or simultaneous route of immunization through the oral cavity, or may be the sole route of immunization without transcutaneous immunization occurring at all. In the latter case, the immunization would be given on intact skin for the express purpose that the animal will ingest the material and become immunized. The antigen, adjuvant and marker may be formulated to contain substances that attract the animal to ingest the immunizing material, encouraging oral immunization. It may hold an advantage to have the animal ingest the material soon after it is taken out of the cold chain. Antigens are generally not stable for long periods and strategies that require ingestion of antigens in bait may be improved by skin-targeted ingestion. It is also within the scope of the present invention, in cases where it is desirable to discourage an animal from licking or otherwise interfering with the presence of the immunization on the surface of the skin, to formulate the antigen, adjuvant and marker to contain substances which are unpleasant to the taste or smell. The application to birds may include application to the cloacal region. With mass immunication such as in chickens, an indicator in the immunizing formulation will allow identification of the treated vs. untreated birds.

In addition to antigen and adjuvant and indicator, the formulation may comprise a vehicle. For example, the formulation may comprise AQUAPHOR (an emulsion of petrolatum, mineral oil, mineral wax, wool wax, panthenol, bisabol, and glycerin as shown in PCT/US97/21324), creams, creams or emulsions containing urea, or other penetration enhancers, emulsions (e.g., aqueous creams), microemulsions, gels, oil-in-water emulsions (e.g., oily creams), anhydrous lipids and oil-in-water emulsions, fats, waxes, oil, silicones, gels, or the same containing excipients and humectants (e.g., glycerol).

The antigen may be derived from a pathogen that can infect the organism (e.g., bacterium, virus, fungus, or parasite), or a cell (e.g., tumor cell or normal cell). The antigen may be a tumor antigen or an autoantigen. The antigen may be an allergen. Chemically, the antigen may be a protein, carbohydrate, glycolipid, glycoprotein, lipid, lipoprotein, phospholipid, polypeptide, or chemical or recombinant conjugate of the above. Antigen may be obtained by recombinant means, chemical synthesis, sonication or other form of disruption or purification from a natural source. Preferred are proteinaceous antigen or conjugates with polysaccharide. Antigen may be at least partially purified in cellfree form. Alternatively, antigen may be provided in the form of a live virus, an attenuated live virus, or an inactivated virus. Indicators may be covalently bonded to antigens, adjuvants, associated with hydrophobic forces, Van der Waals or aggregated, or simply admixed in the solution or vehicle. Indicators may be impregnated into the patch material or other vehicle.

Inclusion of an adjuvant may allow potentiation or modulation of the immune response. The indicator itself may be an adjuvant. For example FITC is a known contact sensitizer and actively fluoresces. Contact sensitizers activate Langerhans cells and may act as adjuvant for coadministered antigens. Moreover, selection of a suitable antigen or adjuvant or indicator may allow preferential induction of a humoral or cellular immune response, specific antibody isotypes (e.g., IgM, IgD, IgA1, IgA2, IgE, IgG1, IgG2, IgG3, and/or IgG4), and/or specific T-cell subsets (e.g., CTL, Th1, Th2, Th3 and/or DTH). Preferably, the adjuvant is an ADP-ribosylating exotoxin or a sub-unit thereof but other adjuvants can be used. Optionally, other means of modifying adjuvants may enhance activation of Langerhans cells, dendritic cells or phagocytic cells and may be used. The term “antigen” as used in the invention, is meant to describe a substance that induces a specific immune response when presented to immune cells of an organism. An antigen may comprise a single immunogenic epitope, or a multiplicity of immunogenic epitopes recognized by a B-cell receptor (i.e., antibody on the membrane of the B cell) or a T-cell receptor. A molecule may be both an antigen and an adjuvant and indicator (e.g., FITC) and, thus, the formulation may contain only one antigen and indicator. The antigen or adjuvant may itself be labeled, such as with a florescent tag or other indicator.

The term “adjuvant” as used in the invention, is meant to describe a substance added to the formulation to assist in inducing an immune response to the antigen.

The term “effective amount” as used in the invention, is meant to describe that amount of antigen which induces an antigen-specific immune response. Such induction of an irnrnune response may provide a treatment such as, for example, immunoprotection, desensitization, immunosuppression, modulation of autoimmune disease, potentiation of cancer immunosurveillance, or therapeutic vaccination against an established infectious disease.

The term “draining lymph node field” as used in the invention means an anatomic area over which the lymph collected is filtered through a set of defined lymph nodes (e.g., cervical, axillary, inguinal, epitrochelear, popliteal, those of the abdomen and thorax).

Without being bound to any particular theory but only to provide an explanation for our observations, it is presumed that the transcutaneous immunization delivery system carries antigen to cells of the immune system where an immune response is induced. The antigen may pass through the normal protective outer layers of the skin (i.e., stratum comeum) and induce the immune response directly, or through an antigen presenting cell population in the epidermis (e.g., macrophage, tissue macrophage, Langerhans cell, dendritic cell, dermal dendritic cell, B lymphocyte, or Kupffer cell) that presents processed antigen to a lymphocyte. Optionally, the antigen may pass through the stratum corneum via a hair follicle or a skin organelle (e.g., sweat gland, oil gland). There is no need to penetrate the skin during immunization and thus, the present invention may be practiced without removal of keratin or the stratum comeum. However, removal of the outer layer of stratum comeum may assist in the immunization. Indicators may be used to stain vital cells and provide guidance to the level of removal of dead cells. Thus, if alcohol swabbing were used for removal of the stratum comeum and a vital stain that stained only living keratinocytes were used, then the vital stain could indicate that suffficient swabbing had been performed. Neither penetration enhancement nor irritation of the outer skin layers is required for immunization but penetration enhancement and irritation may assist in enhancing the immune response.

Transcutaneous immunization with bacterial ADP-ribosylating exotoxins (bAREs) as an example, may target the epidermal Langerhans cell, known to be among the most efficient of the antigen presenting cells (APCs). We have found that bAREs activate Langerhans cells when applied epicutaneously to the skin in saline solution. The Langerhans cells direct specific immune responses through phagocytosis of the antigens, and migration to the lymph nodes where they act as APCs to present the antigen to lymphocytes, and thereby induce a potent antibody response. Although the skin is generally considered a barrier to invading organisms, the imperfection of this barrier is attested to by the numerous Langerhans cells distributed throughout the epidermis that are designed to orchestrate the immune response against organisms invading via the skin. According to Udey (1997), Langerhans cells . . . “comprise all of the accessory cell activity that is present in uninflamed epidermis, and in the current paradigm are essential for the initiation and propagation of immune responses directed against epicutaneously applied antigens.”

The spectrum of more commonly known skin immune responses is represented by contact dermatitis and atopy. Contact dermatitis, a pathogenic manifestation of LC activation, is directed by Langerhans cells which phagocytose antigen, migrate to lymph nodes, present antigen, and sensitize T cells that migrate to the skin and cause the intense destructive cellular response that occurs at affected skin sites (Dahl, 1996; Leung, 1997). Such responses are not generally known to be associated with antigen specific antibodies but may occur in conjunction with TCI. Atopic dermatitis may utilize the Langerhans cell in a similar fashion, but is identified with Th2 cells and is generally associated with high levels of IgE antibody and absence of IgG (Dahl, 1996; Leung, 1997).

Transcutaneous immunization with cholera toxin and related bAREs on the other hand is a novel immune response generally with an absence of findings typical of atopy or contact dermatitis but may have features of either pathology. In some cases induction of pathology may confer an advantage for transcutaneous immunization. The wiqueness of the transcutaneous immune response here is also indicated by the both high levels of antigenspecific IgG antibody, and the type of antibody produced (e.g., IgM, IgG1, IgG2a, IgG2b, IgG3 and IgA) and also may be associated with IgE induction. Transcutaneous immunization could conceivably occur in tandem with skin inflammation if sufficient activation of APCs and T-cells were to occur in a transcutaneous response coexisting with atopy or contact dermatitis.

Transcutaneous targeting of Langerhans cells may also be used in tandem with agents to deactivate their antigen presenting function, thereby modifying immunization or preventing sensitization. Techniques to deactivate Langerhans or other skin immune cells include, for example, the use of anti-inflammatory steroidal or non-steroidal agents (NSAID), cyclophospharnide or other immunosuppressants, interleukin-10,monoclonal antibody to interleukin- 1, ICE inhibitors, anti-TNFa, or depletion via superantigens such as through staphylococcal enterotoxin-A (SEA) induced epidermal Langerhans cell depletion. Similarly, lymphocytes may be immunosupressed before, during or after immunization by administering an immunosupressant such as corticosteroid. For example, hydrocortisone may be coadministered in a patch with the formulation. These additions may be marked by indicators in the immunizing solution, patch, gel emulsion or other delivery vehicle.

Transcutaneous immunization may be induced via the ganglioside GM1 binding activity of CT, LT or sub-units such as CTB. Ganglioside GM1 is a ubiquitous cell membrane glycolipid found in all mammalian cells. When the pentameric CT B sub-unit binds to the cell surface a hydrophilic pore is formed which allows the A to submit across the lipid bilayer (Ribi et al., 1988). Alternatively, TCI may depend on penetration enhancement induced by the presence of CT, LT or other toxins or adjuvants. Zonular toxin may play a particular role in enhancing penetration and inducing an immune response acting as an adjuvant itself, penetration enhancer or in conjunction with an adjuvant.

Efficient immunization can be achieved with the present invention because transcutaneous delivery of antigen may target the Langerhans cell. These cells are found in abundance in the skin and are efficient antigen presenting cells leading to T-cell memory and potent immune responses. Because of the presence of large numbers of Langerhans cells in the skin, the efficiency of transcutaneous delivery may be related to the surface area exposed to antigen and adjuvant. In fact, the reason that transcutaneous immunization is so efficient may be that it targets a larger number of these efficient antigen presenting cells.

We envision the present invention will enhance access to immunization, while inducing a potent immune response. Because transcutaneous immunization does not involve injections and the complications and difficulties thereof, the requirements of trained personnel, sterile technique, and sterile equipment are reduced. The use of an indicator may further simplify the technique of delivery. For example farmers may be able to quite simply immunize the animals on the ear and verify that the immunization has been successfully applied using an indicator. Furthermore, the barriers to immunization at multiple sites or to multiple immunizations are diminished. Immunization by a single application of the formulation is also envisioned.

Processes for preparing a pharmaceutical formulation are well-known in the art, whereby the antigen and adjuvant is combined with a pharmaceutically acceptable carrier vehicle.

Suitable vehicles and their preparation are described, for example, in Remington's Pharmaceutical Sciences, by E. W. Martin. Such formulations will contain an effective amount of the antigen and adjuvant together with a suitable amount of vehicle and indicator in order to prepare pharmaceutically acceptable compositions suitable for administration to a human or animal. The formulation may be applied in the form of a cream, emulsion, gel, lotion, ointment, paste, solution, suspension, or other forms known in the art. In particular, formulations that enhance skin hydration are preferred. There may also be incorporated other pharmaceutically acceptable additives including, for example, diluents, binders, stabilizers, preservatives, and colorings.

Immunization may be achieved using epicutaneous application of a simple solution of antigen and adjuvant impregnated in gauze under an occlusive patch, or by using other patch technologies; creams, gels, immersion, ointments and sprays are other possible methods of to application. The immunization could be given by untrained personnel, and is amenable to self-application. Large-scale field immunization could occur given the easy accessibility to immunization. Additionally, a simple immunization procedure would improve access to immunization by pediatric patients and the elderly, and populations in Third World countries.

Increasing hydration of the stratum comeum will increase the rate of percutaneous absorption of a given solute (Roberts and Walker, 1993). As used in the present invention, penetration enhancer does not include substances such as, for example: water, physiological buffers, and saline solutions which would not perforate the skin. An object of the present invention is to provide a novel means for immunization through intact skin without the need for perforating the epidermis. The transcutaneous immunization system provides a method whereby antigens and adjuvant can be delivered to the immune system, especially specialized antigen presentation cells underlying the skin such as, for example, Langerhans cells. The effectiveness of the penetration enhancement may be verified by using an indicator as described above.

For previous vaccines, their formulations were injected through the skin with needles. Injection of vaccines using needles carries certain drawbacks including the need for sterile needles and syringes, trained medical personnel to administer the vaccine, discomfort from the injection, needle-born diseases, and potential complications brought about by puncturing the skin with the needle. Immunization through the skin without the use of needles (i.e., transcutaneous immunization) represents a major advance for vaccine delivery by avoiding the aforementioned drawbacks. Indicator may further increase the reliability of the technique and enhance the simplicity of monitoring its effectiveness.

Moreover, transcutaneous immunization may be superior to immunization using needles as more immune cells would be targeted by the use of several locations targeting large surface areas of skin. A therapeutically-effective amount of antigen sufficient to induce an immune response may be delivered transcutaneously either at a single cutaneous location, or over an area of intact skin covering multiple draining lymph node fields (e.g., cervical, axillary, inguinal, epitrochelear, popliteal, those of the abdomen and thorax). Such locations close to several different lymphatic nodes at locations all over the body may provide a more widespread stimulus to the immune system than when a small amount of antigen is injected at a single location by intradermal subcutaneous or intramuscular injection. The different locations may have particular formulations that could be demarcated by indicators.

Antigen passing through or into the skin may encounter antigen presenting cells which process the antigen in a way that induces an immune response. Multiple immunization sites may recruit a greater number of antigen presenting cells and the larger population of antigen presenting cells that were recruited would result in greater induction of the immune response. It is conceivable that absorption through the skin may deliver antigen to phagocytic cells of the skin such as, for example, dermal dendritic cells, macrophages, and other skin antigen presenting cells; antigen may also be delivered to phagocytic cells of the liver, spleen, and bone marrow that are known to serve as the antigen presenting cells through the blood stream or lymphatic system. Langerhans cells, dendritic cells, and macrophages may be specifically targeted using β2-macroglobulin bound antigen or Fc receptor conjugated to or recombinantly produced as a protein fusion with adjuvant. Adjuvant may be conjugated to or recombinantly produced as a protein fusion with protein A or protein G to target surface immunoglobulin of B cells. The result would be widespread distribution of antigen to antigen presenting cells to a degree that is rarely, if ever achieved, by current immunization practices. The effective encounter with an APC may be noted by release of an indicator. For example, if the indicator is phagocytosed by the APC, the endocytic vesicle may cause a reaction that can be detected by florescence.

Genetic immunization has been described in U.S. Pat. Nos. 5,589,466, 5,593,972, and 5,703,055. The nucleic acid(s) contained in the formulation may encode the antigen, the adjuvant, or both. The nucleic acid may or may not be capable of replication; it may be non-integrating and non-infectious. The successful replication of the plasmid may be marked by an indicator. Green florescent protein is known to the art and may be encoded on the plasmid with other genes. Successful transfection of an APC may be noted by the presence of florescent product and indicate that the other genes are being produced by the APC or other transfected cell. The nucleic acid may encode a fusion polypeptide comprising antigen and a ubiquitin domain to direct the immune response to a class I restricted response. The nucleic acid may further comprise a regulatory region (e.g., promoter, enhancer, silencer, transcription initiation and termination sites, RNA splice acceptor and donor sites, polyadenylation signal, internal ribosome binding site, translation initiation and termination sites) operably linked to the sequence encoding the antigen or adjuvant. The nucleic acid may be complexed with an agent that promotes transfection such as cationic lipid, calcium phosphate, DEAE-dextran, polybrene-DMSO, or a combination thereof; also, immune cells can be targeted by conjugation of DNA to Fc receptor or protein A/G, or encapsulating DNA in an agent linked to β2-macroglobulin or protein A/G. The nucleic acid may comprise regions derived from viral genomes. Such materials and techniques are described by Kriegler (1990) and Murray (1991).

A similar use of cholera toxin may be employed with non-transcutaneous techniques. Gene gun injection with plasmid DNA and very small amounts of CT or other ADP ribosylating exotoxins may be employed to enhance the immune response to the plasmid products. Indicator genes such as that for green florescent protein, luciferase, may be included in the same plasmid or separate plasmids. It may be that transcutaneously administered CT might target transfected Langerhans cells for activation.

Excipients may be used to enhance the solubility and stability of the antigens to be used in transcutaneous immunization. For example cyclodextrans (CD), which are cyclic carbohydrates, are used to form complexes with hydrophobic drugs, improving their aqueous solubility (Cyclodextrins-enabling excipients: their present and future use in Pharmaceuticals, Diane Thompson, in Critical Reviews in Therapeutic Drug Carrier Systems, 14:100-104 (1997). This technique could be used for hydrophobic antigens. Because it is our expectation that soluble antigens are important for diffusion through the hydrated stratum comeum, excipients that improve the solubility of the antigens would be expected to improve the diffusion through the skin and thus improve the strength and quality of the immune response. Various complexes with different ratios of CD molecules can be formed depending on the size of the antigen and its physicochemical characteristics. Excipients may have other added qualities such as aiding penetration enhancement. Methylated CDs have increased absorption through the transcellular pathway. CDs may be useful for delivery of DNA in the form of naked DNA, lipid complexed DNA such as cationic liposomes, protein antigens such as recombinants, purified proteins, viruses (inactivated or live), synthetic peptides, carbohydrates, conjugates, and other non-protein antigens such a mycolic acid and other TB related antigens.

The present invention is further defined by the following non-limiting example.

### EXAMPLE

Three month old BALB/c mice were transcutaneously immunized and boosted 3 weeks later in a 20 μl dose with:

(a) 25 μg CT (List, #100, lot10050BC) coadministered with 100 μg of DT (List, #151, lot1514A) in 1×PBS (Biowhittaker, #17-512F, lot8M1726) on to the lower back,

(b) 25 μg of CT and 1 μl of red food coloring (contains red FDC#40 & #3; McCormick) coadministered with 100 μg DT and 1 μl of blue food coloring (contains blue FDC #1 & red FDC #40; McCormick) in 1×PBS on to the lower back,

(c) 25 μg of CT and 1 μl of red food coloring in 1×PBS topically applied to the lower back and 100 μg DT and 1 μl of blue food coloring in 1×PBS topically applied to the dorsal neck/upper back, or

(d) 100 μg DT and 1 μl of blue food coloring in 1×PBS topically applied to the lower back.

No mixing of the separated immunogens containing dye in “(c)” were visually apparent during the immunizations.

The results are summarized in Table I.

TABLE 1

| | |
|---|---|
| Experiment # | TC 4.77 |
| ELISA # | EL 330 |
| Date of Primary Immuniz. | 21-May-99 |
| Sample (serum, stool, etc) | SERUM |
| Antibody type (IgG, IgA, etc) | IgG |
| Immunizations and boosts: | |
| 21 May 99, 11 Jun 99, | |
| Bleed dates: 23 Mar 99 (pre-immune), | |
| 26 Jul 99 | |

| | Antigen/Adjuvant | Eartag#s | Collection | | | Assay Values (animals 1-5) | | | | | | Geometric |
|---|---|---|---|---|---|---|---|---|---|---|---|---|
| Group | Dosages | #mice/gp | Date | Detecting | Plate # | 1 | 2 | 3 | 4 | 5 | Units | Mean |
| 1 | CT (25 ug)/DT (100 ug) | 72-76 | pre-immune | CT | 1-2 | <100 | <100 | <100 | <100 | <100 | eu | <100 |
| 2 | CT (25 ug)red/DT (100 ug)blue | 77-81 | pre-immune | CT | 3-4 | <100 | <100 | <100 | <100 | <100 | eu | <100 |
| 3 | CT (25 ug)red neck/ | 82-86 | pre-immune | CT | 5-6 | <100 | <100 | <100 | <100 | <100 | eu | <100 |
| | DT (100 ug)blue lower back | | | | | | | | | | | |
| 4 | DT (25 ug)blue | 87-91 | pre-immune | CT | 7-8 | <100 | <100 | <100 | <100 | <100 | eu | <100 |
| 1 | CT (25 ug)/DT (100 ug) | 72-76 | 26-Jul-99 | CT | 1-2 | 3255 | 2548 | 706 | 2771 | 12608 | eu | 2898 |
| 2 | CT (25 ug)red/DT (100 ug)blue | 77-81 | 26-Jul-99 | CT | 3-4 | 603 | <100 | 503 | 1826 | 941 | eu | 482 |
| 3 | CT (25 ug)red neck/ | 82-86 | 26-Jul-99 | CT | 5-6 | 48660 | <100 | 525 | 994 | 34404 | eu | 2128 |
| | DT (100 ug)blue lower back | | | | | | | | | | | |
| 4 | DT (25 ug)blue | 87-91 | 26-Jul-99 | CT | 7-8 | <100 | <100 | <100 | <100 | <100 | eu | <100 |
| 1 | CT (25 ug)/DT (100 ug) | 72-76 | pre-immune | CT | 1-2 | <100 | <100 | <100 | <100 | <100 | eu | <100 |
| 2 | CT (25 ug)red/DT (100 ug)blue | 77-81 | pre-immune | CT | 3-4 | <100 | <100 | <100 | <100 | <100 | eu | <100 |
| 3 | CT (25 ug)red neck/ | 82-86 | pre-immune | CT | 5-6 | <100 | <100 | <100 | <100 | <100 | eu | <100 |
| | DT (100 ug)blue lower back | | | | | | | | | | | |
| 4 | DT (25 ug)blue | 87-91 | pre-immune | CT | 7-8 | <100 | <100 | <100 | <100 | <100 | eu | <100 |
| 1 | CT (25 ug)/DT (100 ug) | 72-76 | 26-Jul-99 | DT | 1-2 | <100 | 127 | <100 | 542 | 770 | eu | 168 |
| 2 | CT (25 ug)red/DT (100 ug)blue | 77-81 | 26-Jul-99 | DT | 3-4 | <100 | <100 | 306 | <100 | 348 | eu | 106 |
| 3 | CT (25 ug)red | 82-86 | 26-Jul-99 | DT | 5-6 | 2119 | <100 | <100 | <100 | 6267 | eu | 278 |
| | top/DT (100 ug)blue bottom | | | | | | | | | | | |
| 4 | DT (25 ug)blue | 87-91 | 26-Jul-99 | DT | 7-8 | <100 | <100 | <100 | <100 | <100 | eu | <100 |

Although the present invention has been described in detail with reference to its presently preferred embodiments, it will be understood by those of ordinary skill in the art that various modifications and improvements to the present invention are believed to be apparent to one skilled in the art. It is intended that the scope of the invention be defined by the following claims.

What I claim is:

1. A transcutaneous immunization system, for immunizing a subject comprising:
a transcutaneous immunization formulation; said immunization formulation comprising at least one antigen, and
a marking formulation; said marking formulation comprising at least one chemical indicator, the presence of said indicator serving to provide a readily discernable indication of the use of said immunization formulation,
wherein said transcutaneous immunization system provides an antigen specific immune response in the subject following the application of said system to intact skin of said subject.

2. The immunization system of claim 1, wherein said immunization formulation further comprises an adjuvant.

3. The immunization system of claim 1, wherein said chemical indicator also acts as an adjuvant.

4. The immunization system of claim 1, wherein said indicator further provides an indication of the length of time to which said subject has been exposed to said immunization formulation.

5. The immunization system of claim 4, wherein said length of time is determinable by said indicator taking on a color, changing color, or loosing the appearance of a color.

6. The immunization system of claim 4, wherein said antigen is two or more specific antigens and said at least one indicator provides a specific, distinctive, discernable indication for each of said two or more specific antigens.

7. The immunization system of claim 1, wherein said indication is discernable by visual or olfactory senses.

8. The immunization system of claim 1, wherein said indicator is formulated to deactivate said antigen after a specific time of exposure of said antigen to said subject.

9. The immunization system of claim 1, wherein said indicator provides an indication of the degree of hydration of the skin surface of the subject.

10. The immunization system of claim 2, wherein said indicator is associated with said antigen or adjuvant by covalent bonding, hydrophobic forces, Van der Waals or aggregated, or admixed.

11. The immunization system of claim 10, wherein said indicator is a florescent tag.

12. The immunization system of claim 1, wherein said indicator provides a discemable indication of specific antigen present in said immunization formulation, the time span of the immunization application, or the effectiveness of said immunization application.

13. The immunization syst em of claim 1, wherein said indicator is selected from the group consisting of dye, ink, vital stain, Evans blue, paint, natural colorings, oxides, chlorophyll, charcoals, chalks, powders, pH indicators, peroxidase triggered or enzyme triggered compound, florescent compound, radioactive tag, attached to beads, and gold particles.

14. The immunization system of claim 1, further comprising an applicator, said applicator being selected from the group consisting of a patch, cream, ointment, gel, and any combination thereof.

15. The immunization system of claim 10, wherein said applicator is a patch.

16. The immunization system of claim 15, wherein said patch comprises a controlled release reservoir, a matrix, or a rate controlling membrane for stepped release of said immunization formulation.

17. The immunization system of claim 1, wherein said subject is an animal.

18. The immunization system of claim 17, wherein said system is deliverable by means of an air gun.

19. The immunization system of claim 17, wherein said system is formulated to induce said animal to lick or smell said formulation from the application site on the skin.

20. The immunization system of claim 17, wherein said system is formulated to inhibit the animal from licking or smelling said formulation from the application site on the skin.

21. A method for eliciting an immune response from a subject by the application of an immunization system, said method comprising:
applying an immunization formulation to at least one application site on the intact skin of a subject, said immunization formulation comprising at least one antigen; and
applying a marking formulation to said at least one application site,
wherein said method provides an antigen specific immune response in the subject following the application of said system to intact skin of said subject.

22. The method of claim 21, further comprising pre-treating said intact skin by tape stripping, washing, or swabbing with alcohol or acetone.

23. The method of claim 21, further comprising applying said method to more than one application site on said subject.

24. The method of claim 21, further comprising applying said method to a site overlying more than one draining lymph node.

25. The method of claim 21, wherein said antigen is selected from the group consisting of pathogen derived antigen, tumor antigen and autoantigen.
